(19) United States
(12) Patent Application Publication
(10) Pub. No.: US 2022/0153932 A1
(43) Pub. Date: May 19, 2022

(54) PREPOLYMERS OF 1,1-DICARBONYL SUBSTITUTED-1-ALKENE, METHODS TO MAKE THEM, POLYMERS MADE FROM THEM AND METHODS TO MAKE THE POLYMER

(71) Applicant: Sirrus, Inc., Loveland, OH (US)

(72) Inventors: J. Taylor Goodrich, Loveland, OH (US); Alexander Y. Polykarpov, Brunswick, OH (US)

(21) Appl. No.: 17/405,554

(22) Filed: Aug. 18, 2021

Related U.S. Application Data
(60) Provisional application No. 63/115,159, filed on Nov. 18, 2020.

(51) Int. Cl. C08G 75/045; C07C 323/52; C07C 319/18

(52) U.S. Cl. CPC C08G 75/045; C07C 319/18; C07C 323/52

(57) ABSTRACT

A liquid 1,1-dicarbonyl substituted 1-alkene prepolymer is formed by reacting a multifunctional 1,1-dicarbonyl substituted-1-alkene and a polythiol or monothiol at a ratio of thiols/carbon-carbon double bonds of 0.001 to 0.5 in the presence of 0.1 parts per million (ppm) to 100 ppm by weight of strong acid, polythiol/monothiol and 1,1-dicarbonyl substituted-1-alkene. Further ingredients, such as other monomers, fillers, and rheological modifiers may be added to form polymerizable compositions. The prepolymer or polymerizable compositions may be addition polymerized to form polymer articles.

### FIELD

The invention relates to 1,1-dicarbonyl substituted-1-alkenes prepolymers. In particular, the invention relates to forming liquid 1,1-dicarbonyl substituted-1-alkene prepolymers formed using polythiols.

### BACKGROUND

1,1-dicarbonyl substituted-1-alkenes have been known for some time and described in U.S. Pat. Nos. 2,330,033; 3,221,745 and 3,523,097; 3,197,318; 4,056,543 and 4,160,864. Despite this the 1,1-dicarbonyl substituted-1-alkenes have not seen substantial commercialization.

1,1-dicarbonyl substituted-1-alkenes compounds rapidly polymerize at room temperature under mild conditions in the presence of nucleophilic or basic initiating species, which render them both useful, as well as, present problems with their stability and workability. More recently, processes to produce the 1,1-dicarbonyl substituted-1-alkenes that solved some of the stability issues were described in U.S. Pat. Nos. 8,609,885; 8,884,405; U52014/0329980; and US 2015/0073110; all incorporated herein by reference in their entirety for all purposes. However, due to their high reactivity it is still often challenging to prepare a single component system. While curable one component compositions of methylene malonates based on latent or microencapsulated initiators have been described (e.g., U.S. Pat. No. 9,181,365) such approaches may limit the final polymer characteristics achieved.

The 1,1-dicarbonyl substituted-1-alkenes have been used for certain applications. Recently, some methods were described attempting to further broaden the utility of 1,1-dicarbonylsubstituted-1-alkenes for preparation of polymers with improved properties. For example, copolymerizing two or more 1,1-dicarbonyl substituted-1-alkene monomers having substantially different homopolymer glass transition temperatures (see, for example, U.S. Pat. No. 9,315,597) have been described to produce polymers with a broad range of Tg. The 1,1-dicarbonyl substituted-1-alkenes have been reacted with diols to form polyester macromere, which are then subsequently polymerized to form coatings and the like. (U.S. Pat. No. 9,617,377). The 1,1-dicarbonyl substituted-1-alkenes have been UV polymerized with other radically polymerizable monomers and oligomers in the presence of a UV initiator (e.g., copending U.S. provisional application 62/987,507).

Accordingly, it would be desirable to provide a composition comprised of a 1,1-dicarbonyl substituted-1-alkene that enables or improves one or more characteristic such as shelf life stability, increased flexibility in tailoring the properties of the polymer expanding use of the 1,1-dicarbonyl substituted-1-alkenes in applications such as adhesive, biodegradable polymer applications and biological applications (e.g., grafting scaffolds).

### SUMMARY

It has been discovered that a 1,1-dicarbonyl substituted-1-alkene may react with thiols under mild conditions (e.g., Michael addition) that does not cause in essence any addition polymerization of the alkenes of the 1,1-dicarbonyl substituted-1-alkene allowing the formation of a prepolymer having improved stability over the 1,1-dicarbonyl substituted-1-alkene so long as the reaction is performed in the presence of a strong acid at low concentrations.

A first aspect of the invention is a method of forming a 1,1-dicarbonyl substituted-1-alkene prepolymer comprising:

a. mixing a multifunctional 1,1-dicarbonyl substituted-1-alkene having an average functionality of greater than 1 to 10 and a polythiol having an average functionality greater than 1 to about 6 at a ratio of thiols/carbon-carbon double bonds of the alkene of 0.001 to 0.5 to form a mixture, b. allowing the multifunctional 1,1-dicarbonyl substituted-1-alkene and polythiol to react in the presence of a strong acid at a concentration of 0.1 parts per million to 100 parts per million by weight of the mixture to form the 1,1-dicarbonyl substituted-1-alkene prepolymer. Ambient conditions (˜20° C. to ˜25° C.) are sufficient and desirable for carrying out the reaction, even though other temperatures may be used, excess heating may not be desirable because of possible addition polymerization of the carbon-carbon double bond.

Multifunctional 1,1-dicarbonyl substituted-1-alkene means there is more than 1 carbon-carbon double bond in the 1,1-dicarbonyl substituted-1-alkene. It is understood that the average functionality of the 1,1-dicarbonyl substituted-1-alkene is greater than 1 to about 10. Illustratively, the multifunctional 1,1-dicarbonyl substituted-1-alkene is an oligomer of two or more monomeric 1,1-dicarbonyl substituted alkene having one alkene as described below.

A second aspect of the invention is a 1,1-dicarbonyl substituted-1-alkene prepolymer comprised of the reaction product of a multifunctional 1,1-dicarbonyl substituted-1-alkene and a polythiol, wherein the prepolymer is a liquid having an amount of a strong acid from 0.1 ppm to 100 ppm (parts per million) by weight of the prepolymer and strong acid.

A third aspect of the invention is a polymerizable composition comprised of the prepolymer of aspect 2 of the invention and a further ingredient comprised of one or more of a filler, plasticizer, dye, an addition polymerizable monomer or oligomer that is different than the prepolymer, a polyfunctional Michael addition compound other than one containing a thiol, ultraviolet stabilizers, antioxidants, catalyst, or rheological modifiers.

A fourth aspect of the invention is a method of forming a functionalized 1,1-dicarbonyl substituted-1-alkene prepolymer comprising:

a. mixing a multifunctional 1,1-dicarbonyl substituted-1-alkene having an average functionality of greater than 1 to 10 and a thiol having an additional functional group at a ratio of thiols/carbon-carbon double bonds of the alkene of 0.001 to 0.5 to form a mixture,

b. allowing the multifunctional 1,1-dicarbonyl substituted-1-alkene and polythiol to react in the presence of a strong acid at a concentration of 0.1 parts per million to 100 parts per million by weight of the mixture to form the functionalized 1,1-dicarbonyl substituted-1-alkene prepolymer. The additional functional group of the thiol (monothiol) is a functional group that does not react with the 1,1-dicarbonyl substituted-1-alkene or any other groups at the reaction conditions used to make the functionalized prepolymer. The groups may be any that may then be reacted upon polymerization of the alkenes to form a polymer from the prepolymer. Examples of such functional groups include other addition reactive bonds, alcohols, carboxylic acid and the like that do not catalyze or react at the conditions to form the functionalized prepolymer.

A fifth aspect of the invention is a functionalized 1,1-dicarbonyl substituted-1-alkene prepolymer comprised of the reaction product of a multifunctional 1,1-dicarbonyl substituted-1-alkene and a thiol having an additional functional group, wherein the prepolymer is a liquid having an amount of a strong acid from 0.1 ppm to 100 ppm (parts per million) by weight of the prepolymer and strong acid. A polymerizable composition may be made using the functionalized 1,1-dicarbonyl substituted-1-alkene prepolymer in the same manner as described for the third aspect.

The prepolymers of the invention have a sufficient amount of the carbon carbon double bonds reacted with the thiol groups of the polythiol such that the prepolymer has desired stability and forms desired polymers formed therefrom. “Stability” in this context generally means that the prepolymer or polymerizable compositions made therefrom have a shelf life of at least 3 months, 4 months or even 6 months at typical ambient temperatures (20° C. to 25° C.) in that the viscosity does not increase so much that the prepolymer or composition made therefrom becomes unusable (e.g., cannot be adequately pumped, cast or molded). Typically, this means the viscosity does not increase more than about 50 percent during the stated period. To measure the stability an accelerated test may be performed, for example, by maintaining the prepolymer at an elevated temperature for 30 days (e.g., 50° C.) and testing the viscosity prior to and after the heat treatment. Desirably the viscosity does not increase more than 20% in the elevated temperature test Viscosity may be measured using a Brookfield viscometer at a fixed rpm (e.g., 20) rpm and appropriate spindle (e.g., #6 spindle) at ambient conditions (e.g., 23° C.±2° C.).

A sixth aspect of the invention is an article comprised of a polymerized prepolymer of the second aspect or polymerized composition of the third aspect of the invention. The article may be any article such as any article that has been molded by known methods such as cast molding, injection molding, blow molding or the like. The article may be, for example, a coating, adhesive, caulk, sealant, ink, or additive manufactured article (e.g., extrusion type additive manufactured articles).

A seventh aspect of the invention is an article comprised of a polymerized prepolymer of the fifth aspect of the invention. The article may be any article as described for the sixth aspect of the invention.

The properties of the polymer made from the prepolymer or polymerizable composition may vary widely depending on the particular multifunctional 1,1-dicarbonyl substituted alkene used or mixtures of them used, the amount and type(s) of polythiol used to form the prepolymer as well as any additives or other comonomers added to the prepolymer. This allows for the making of articles that may vary from elastic to rigid as well as have a wide range of glass transition temperatures (Tg). As such, the polymer, compositions to make polymer and articles made therefrom may be suitable for a myriad of applications as previously described.

### DETAILED DESCRIPTION

The explanations and illustrations presented herein are intended to acquaint others skilled in the art with the invention, its principles, and its practical application. The specific embodiments of the present disclosure as set forth are not intended to be exhaustive or limit the scope of the disclosure.

One or more as used herein means that at least one, or more than one, of the recited components may be used as disclosed. It is understood that the functionality of any ingredient or component may be an average functionality due to imperfections in raw materials, incomplete conversion of the reactants and formation of by-products. The 1,1-dicarbonyl substituted-1-alkene compounds are for convenience referred to as “1,1-dicarbonyl alkene(s)” interchangeably. The 1,1-dicarbonyl substituted-1-alkene prepolymer are for convenience referred to as “prepolymer(s)”.

The method to form the prepolymers comprises mixing a 1,1-dicarbonyl substituted-1-alkene with a polythiol or monothiol as the case may be to form a reaction mixture. The mixing may be performed under any conditions or apparatus known in the art for mixing two components. In some embodiments, a solvent may be used to dissolve the 1,1-dicarbonyl substituted alkene and polythiol to form the mixture. Useful solvents may include any solvent that dissolves the 1,1-dicarbonyl substituted alkenes and the polythiol. Solvents may be any polar solvent, a protic solvent, water or combination thereof. Solvents may, for example, include ethers, ketones and the like so long as they are not a terminated with a polythiol or interfere with the reaction of the thiol(s) of the polythiol or monothiol with the alkene of the 1,1-dicarbonyl substituted-1-alkene.

To ensure the prepolymer does not gel and remains a liquid to allow, for example, the shaping of the prepolymer or polymerizable compositions made therefrom to form an article, the amount of polythiol or monothiol should not be too great to cause gelling or instability. Typically, the amount of polythiol or monothiol is such that there are 0.001 to 0.5 thiols/carbon-carbon double bonds (C═C). Desirably, the thiol/C═C ratio is about 0.01, 0.02, 0.05, 0.1 to 0.4, 0.3 or 0.25. The average polythiol functionality is typically from greater than 1 to about 6. Desirably, the average functionality of the polythiol is from about 1.5, to about 5, 4, 3, or 2.5. Nominally, it is desirable for the polythiol to have, in essence, 2 thiol groups. The particular thiol/C═C ratio that is suitable may be influenced by the functionality of the polythiol. For example, higher functionality polythiols may require a lower thiol/C═C ratio to have the desired stability compared to lower functionality polythiols. In the case of the monothiol having a functional group, the thiol/alkene ratio may have a wider range without gelling and still having suitable rheological behavior.

The polythiol may be any useful polythiol such as those known in the art. The polythiol may be an oligomer or polymer (e.g., random, branched, block or any combination therof). The molecular weight of the polythiol may be any useful molecular weight. Illustratively, the weight average molecular weight may be from about 200, 300, 500, 1000, 2000 to 250,000, 100,000, 75,000, 50,000, 30,000, or 20,000 g/moles.

The monothiol having an additional functional group may be any useful monothiol such as those known in the art and may be of similar structure and Mw described above. The additional functional group, to reiterate, may be any functional group that does not interfere with the reaction between the thiol and alkenes of the multifunctional 1,1-dicarbonyl substituted-1-alkene. Examples include oligomers or polymers as described below for the polythiol so long as there is at least one additional functional group. In an embodiment the monothiol having a functional group has one functional group and it desirably is a terminal group.

Illustratively, the polythiol may be an oligomer or polymer having a polymer backbone comprised of an aromatic, aliphatic, combination of aliphatic and aromatic, polyether, polyester, polythioether, polysulfide or combination thereof. Examples of suitable polythiols may be those known in the art. For example, the polythiol may be a terminated polyether or polyester such as those available from Nanosoft Polymers (Lewisville, N.C.). The polythiol maybe a polysulfide available under the tradename THIOKOL LP from Toray Fine Chemicals Co. Ltd. Japan. The polythiol may be mercaptan terminated polymers such as those known for use in curing epoxy systems, with a commercial example being those available under the tradenames CAPCURE and GABEPRO available from Gabriel, Akron, Ohio. The polythiol may be a liquid polysulfide prepolymer end-capped by SH functional end groups such as those available under the tradename THIOPLAST G from Nouryon Functional Chemicals GmBH.

The monothiol may be a heteroterminated linear polyether or polyester in which one terminal group is the thiol and the other terminal group is the functional group such as an alcohol, carboxylic acid (e.g., terminated with lactic acid), deoxyribonucleic acid, peptide, cholesterol, biotin, folate, and the like such as available from Nanosoft Polymers described above.

The multifunctional 1,1-dicarbonyl alkene has more than one alkene group. The 1,1-dicarbonyl-1-alkene are compounds wherein a central carbon atom is doubly bonded to another carbon atom to form a double bond. The central carbon atom is further bonded to two carbonyl groups. Each carbonyl group is bonded to a hydrocarbyl group through a direct bond or an oxygen atom. Where the hydrocarbyl group is bonded to the carbonyl group through a direct bond, a ketone group is formed. Where the hydrocarbyl group is bonded to the carbonyl group through an oxygen atom, an ester group is formed. The multifunctional 1,1-dicarbonyl-1-alkene has at least two alkenes as described.

Exemplary multifunctional 1,1-dicarbonyl alkenes are illustrated by the formula:

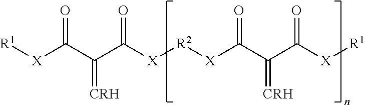

wherein R¹ and R² are described below; X is, separately in each occurrence, an oxygen atom or a direct bond; n is an integer of 1 or greater to any useful amount such as a polymer of 1,000 or 10,000 Daltons or more to typically at most about 1,000,000 or 100,000 and R is hydrogen or a hydrocarbyl group having 1 to 30 carbons, so long as at least one R is hydrogen (i.e., ═CH₂) and preferably every R is hydrogen. Typically, n is 1 or 2 to 20 or 10.

The hydrocarbyl groups (e.g., R¹ and R²), each may comprise straight or branched chain alkyl, straight or branched chain alkyl alkenyl, straight or branched chain alkynyl, cycloalkyl, alkyl substituted cycloalkyl, aryl, aralkyl, or alkaryl. The hydrocarbyl group may optionally include one or more heteroatoms in the backbone of the hydrocarbyl group. The hydrocarbyl group may be substituted with a substituent that does not negatively impact the ultimate function of the 1,1-dicarbonyl alkene or the polymer prepared from the 1,1-dicarbonyl alkene. Preferred substituents include alkyl, halo, alkoxy, alkylthio, hydroxyl, nitro, cyano, azido, carboxy, acyloxy, and sulfonyl groups. More preferred substituents include alkyl, halogen, alkoxy, allylthio, and hydroxyl groups. Most preferred substituents include halogen, alkyl, and alkoxy groups.

As used herein, alkaryl means an alkyl group with an aryl group bonded thereto. As used herein, aralkyl means an aryl group with an alkyl group bonded thereto and include alkylene bridged aryl groups such as diphenyl methyl groups or diphenyl propyl groups. As used herein, an aryl group may include one or more aromatic rings. Cycloalkyl groups include groups containing one or more rings, optionally including bridged rings. As used herein, alkyl substituted cycloalkyl means a cycloalkyl group having one or more alkyl groups bonded to the cycloalkyl ring.

The hydrocarbyl groups may include 1 to 30 carbon atoms, 1 to 20 carbon atoms, or 1 to 12 carbon atoms. Hydrocarbyl groups with heteroatoms in the backbone may be alkyl ethers having one or more alkyl ether groups or one or more alkylene oxy groups. Alkyl ether groups may be ethoxy, propoxy, and butoxy. Such compounds may contain from about 1 to about 100 alkylene oxy groups, about 1 to about 40 alkylene oxy groups, about 1 to about 12 alkylene oxy groups, or about 1 to about 6 alkylene oxy groups.

One or more of the hydrocarbyl groups (e.g., R¹, R², or both) may include a C₁-C₁₅ straight or branched chain alkyl, a C₁-C₁₅ straight or branched chain alkenyl, a C₅-C₁₈ cycloalkyl, a C₆-C₂₄ alkyl substituted cycloalkyl, a C₄-C₁₈ aryl, a C₄-C₂₀ aralkyl, or a C₄-C₂₀ aralkyl. The hydrocarbyl group may include a C₁-C₈ straight or branched chain alkyl, a C₅-C₁₂ cycloalkyl, a C₆-C₁₂ alkyl substituted cycloalkyl, a C₄-C₁₈ aryl, a C₄-C₂₀ aralkyl, or a C₄-C₂₀ aralkyl.

Alkyl groups may include methyl, propyl, isopropyl, butyl, tertiary butyl, hexyl, ethyl pentyl, and hexyl groups. More preferred alkyl groups include methyl and ethyl. Cycloalkyl groups may include cyclohexyl and fenchyl. Alkyl substituted groups may include menthyl and isobornyl, norbornyl as well as any other bicyclic, tricyclic or polycyclic structure.

Hydrocarbyl groups attached to the carbonyl group may include methyl, ethyl, propyl, isopropyl, butyl, tertiary, pentyl, hexyl, octyl, fenchyl, menthyl, and isobornyl, cyclic, bicyclic or a tricyclic group such as cyclohexyl, norbornyl, or tricyclodecanyl.

In exemplary embodiments R² may be, separately in each occurrence, straight or branched chain alkyl, straight or branched chain alkenyl, straight or branched chain alkynyl, cycloalkyl, alkyl substituted cycloalkyl, aryl, aralkyl, or alkaryl, wherein the hydrocarbyl groups may contain one or more heteroatoms in the backbone of the hydrocarbyl group and may be substituted with a substituent that does not negatively impact the ultimate function of the compounds or polymers prepared from the compounds. Exemplary substituents may be those disclosed as useful with respect to R1. In certain embodiments R² may be, separately in each occurrence, C₁₋₁₅ straight or branched chain alkyl, C₂₋₁₅ straight or branched chain alkenyl, C₅₋₁₈ cycloalkyl, C₆₋₂₄ alkyl substituted cycloalkyl, C₄₋₁₈ aryl, C₄₋₂₀ aralkyl or C₄₋₂₀ aralkyl groups. In certain embodiments R² may be separately in each occurrence C₁₋₈ straight or branched chain alkyl, C₅₋₁₂ cycloalkyl, C₆₋₁₂ alkyl substituted cycloalkyl, C₄₋₁₈ aryl, C₄₋₂₀ aralkyl or C₄₋₂₀ alkaryl groups.

In an embodiment, X is O and R² is the residue of a diol, wherein a polyester is formed. The polyesters may be formed from any suitable 1,1-dicarbonyl alkene such as the malonates described above and as described in U.S. Pat. No. 9,969,822 from col. 19, line 49 to col. 20, line 3 and a polyol incorporated herein by reference. Examples of suitable polyol include, for example, those described in U.S. Pat. No. 9,969,822 from col. 20, line 18 to col. 21, line 26, incorporated herein by reference. Examples of diols may include ethylene diol 1,3-propylene diol, 1,2 propylene diol, 1-4-butanediol, 1,2-butane diol, 1,3-butane diol, 2,3-butane diol, 1,5-pentane diol, 1,3- and 1,4-cyclohexanedimethanols or combinations thereof. Examples of triols may include 1,2,3-propane triol, 1,2,3-butane triol, trimethylolpropane, 1,2,4-butane triol or combination thereof. Likewise, the polyol may be even higher functional, for example, di(trimethylolpropane), pentaerythritol, dipentaerythritol or combination thereof. Any combination of polyols such as multiple diols, triols, tetraols, pentaols, hexaols or mixtures thereof may be used.

To form the prepolymers, the reaction mixture is reacted in the presence of a strong acid. The strong acid may be any inorganic acid or organic acid, for example that has a pKa of less than about 3. The pKa may be about −12 to 3. Examples of organic acids include methane sulfonic acid (MSA) or para toluene sulfonic acid (PTSA). The inorganic acid may be hydrochloric acid, nitric acid, sulphuric acid or combination thereof. Desirably the strong acid is an organic acid with MSA being particularly suitable. The strong acid may be added at any time to the reaction mixture and may be included with the multifunctional 1,1-dicarbonyl alkene prior to mixing with the polythiol.

The strong acid is provided in an amount from 0.1 ppm to 100 ppm by weight of the mixture of the 1,1-dicarbonyl alkene, polythiol and strong acid mixture. It has been discovered that an excess amount of strong acid may lead to instability of the prepolymer, which may be due to anionic initiation occurring due to anions forming due to rescission of the alkene thiol Michael addition bond. Too low a concentration may also lead to instability or lack of complete thiol-alkene reactions. Desirably, the amount strong acid is from about 1 ppm, 5 ppm or 10 ppm to about 95 ppm, 90 ppm, 85 ppm or 75 ppm of the mixture.

The reaction desirably is performed under mild conditions such as ambient conditions (e.g., ˜1 atmosphere and temperature of 20° C. to 25° C.). The temperature may be elevated, but generally is not desired, because it is not necessary and may lead to undesired reactions such as causing a reaction with the additional functional group of the monothiol. A lower temperature than ambient may be useful, but are generally not necessary.

The amount of carbon-carbon double bonds that have been reacted in the prepolymer may be determined using HNMR. The amount of carbon-carbon double bonds consumed in an 1,1-dicarbonyl-1-alkene oligomer (e.g., methylene malonate polyester) described below may be determined using the alkene number. The alkene number can be calculated from quantitative HNMR of methylene malonate polyesters, where a sample of the polyester with HMDSO standard is analyzed by HNMR spectroscopy in suitable deuterated solvent (i.e. CDCl₃). Hexamethyldisiloxane (M.W. HMDSO, molecular weight is 162 g/mol) can be used as a standard when acquiring the HNMR spectra.

The calculation for alkene value (mmol/g), which is the mmol of methylene malonate per 1 g of the 1,1-dicarbonyl-1-alkene polyester oligomer, can be performed using the following equation;

$$Alkene Value = \frac{Alkene CH 2 Peak Area}{Peak Area HMDSO} \times \frac{18}{2} \times \frac{mHMDSO}{m} \times \frac{1000}{M . W . HMDSO} ,$$

where the Alkene CH₂ Peak Area is measured around 6.3 ppm and is a combined area for all methylene signals in this area; Peak Area HMDSO is measured as the reference at 0 ppm, 18 is the number of hydrogen nuclei in HMDSO and 2 is the number of hydrogen nuclei of the methylene malonate methylene group (CH₂); m HMDSO is the weight of HMDSO in the sample, m is the weight of the polyester.

To calculate the alkene number, theoretical alkene value (mmol/g), which is the mmol of methylene malonate methylene per 1 g of the 1,1-dicarbonyl-1-alkene polyester described below assuming no Michael adduct, is also needed. The following is the explanation about how to calculate theoretical alkene value. The polyester with no Michael adduct derived from the reaction between methylene malonate monomer (ROOC—C(═CH2)-COOR′) and diol (HO—X—OH) can be described as ROOC—C(═CH2)(-COO—X—OOC—C(═CH2)-)₍ₙ₋₁₎COOR′, where n is the number of the methylene malonate methylene functional groups per number average molecular weight of the polyester (Mn.polyester) assuming no Michael adduct. Because the polyester can be regarded as the combination of three parts, which are ROOC—C(═CH2)-COOR′, X₍ₙ₋₁₎ and (OOC—C(═CH2)-COO)₍ₙ₋₁₎, the number average molecular weight of the polyester (Mn) can be expressed by the following equation; Mn=M.W.monomer+M.W.X x(n−1)+114×(n−1), where M.W.monomer is the molecular weight of the monomer and M.W.X is the molecular weight of the linker of diol (X). By transforming this equation, n can be expressed by the following equation;

$$n = \frac{Mn . polyester - M . W . monomer + 114 + M . W . X}{114 + M . W . X} .$$

Theoretical alkene value (mmol/g) can be expressed as (n/Mn.polyester)×1000. Based on the equation above, this can be calculated as follows; Theoretical alkene value:

$$( \frac{m mol}{g} ) = \frac{Mn . polyester - M . W . monomer + 114 + M . W . X}{114 + M . W . X} \times \frac{1000}{Mn . polyester} .$$

And the Alkene number can be calculated as;

$$Alkene number = \frac{Alkene Value}{Theoretical Alkene Value} \times 100.$$

This can then be used to determine the percentage of alkenes that have been converted for the oligomers of the 1,1-dicarbonyl-1-alkenes. Generally, the alkene number of the prepolymer is from about 50, 60, 70, 75 to 99.9, 99, 95 or 90.

The prepolymers are liquid at ambient conditions (i.e., ˜20 to 25° C.). The prepolymer desirably exhibits a viscosity, which facilitates formulation of a polymerizable composition having desirable characteristics to make coatings, adhesives, caulks, molded articles and additive manufactured articles. Typically, the viscosity of the prepolymer is at most about 100,000 centipoise (cps), 50,000 cps, 30,000 to about 10, 100 or 1000 cps. The viscosity may be measured at a single shear rate and at ambient conditions such as when determining stability described herein. The rheological behavior may be Newtonian or non-Newtonian.

The prepolymer may have any useful Mw, but generally is about 500 g/moles, 1000, 2000, 5000 to about 100,000 g/moles, 80,000 g/moles, 50,000 or 30,000 g/moles.

The 1,1-dicarbonyl substituted alkene prepolymer or functionalized prepolymer or combination thereof may be combined with further components to form a polymerizable composition. The further components may be one or more dyes, pigments, toughening agents, rheology modifiers, fillers, reinforcing agents, thickening agents, opacifiers, inhibitors, fluorescence markers, thermal degradation reducers, thermal resistance conferring agents, surfactants, wetting agents, or stabilizers. For example, thickening agents and plasticizers such as vinyl chloride terpolymer (comprising vinyl chloride, vinyl acetate, and dicarboxylic acid at various weight percentages) and dimethyl sebacate respectively, can be used to modify the viscosity, elasticity, and robustness of a system. In certain embodiments, such thickening agents and other compounds can be used to increase the viscosity of a polymerizable system comprised of the polymerizable composition from 1 to 3 cPs to about 100,000 cPs, or more.

In a particular embodiment, the polymerizable composition when used for caulks, sealants, adhesives and extrudates useful for additive manufacturing, the prepolymer may have a viscosity of about 100,000 centipoise (100 Pas), 50,000 centipoise (50 Pas) or 30,000 centipoise (30 Pas) or less to 1,000 centipoise (1 Pas) or greater. The viscosity may be determined using a Brookfield viscometer using #5 or #6 spindle at a constant rpm. In another embodiment the polymerizable composition may display non-newtonian rheology. In particular, the rheology may be shear thinning and may also display a yield stress.

In an embodiment the polymerizable composition is shear thinning and may display a yield stress. A useful indicative low shear measurement is one in which the viscosity is measured using a Brookfield viscometer using a number 5 spindle at the lowest rpm or using a AR2000 Rheometer available from TA Instruments, New Castle, Del. with a continuous flow method where a 4 degrees cone plate of 20 mm diameter is used at 25 degrees C. along with 152 micrometer gap and a shear sweep from 1 to 150 s⁻¹. The viscosity in centipoise at low shear is taken at a shear rate of 5 s⁻¹.

Likewise, the composition desirably has a lower viscosity at higher shear (i.e., is shear thinning) to aid in the ease of dispensing. Generally, it is desirable for the material to have a viscosity at 100 s⁻¹ that is at least 2, 3, 5, 10 or even 20 or more times less than at a shear rate of 5 s⁻¹.

In a particular embodiment, it is desirable for the polymerizable composition to have a yield stress prior to flowing, which aids in the retention of the cross-sectional shape imparted upon dispensing through the nozzle opening (such as when applying a bead of caulk). The yield stress is characterized by measuring G′, the storage modulus, using a rheometer. In measuring the yield stress, the material is first mixed at high shear such as mixing in a container with paddle blades rotating at 200 rpm for about 1 minute. The material is then placed in a rheometer (e.g., AR2000 rheometer from TA Instruments) and an oscillatory stress sweep from 10 to 10,000 Pa at a frequency of 0.1 Hz is performed accordingly. A suitable measuring device geometry is a 25 mm parallel plate having a gap of about 1,000 micrometers. Prior to performing the sweep, a dynamic pre-shear is used to mitigate any residual normal force caused by setting the gap of the parallel plate. A suitable dynamic pre-shear consists of a 0.01 rad displacement at a frequency of 1 Hz for about 1 minute.

Generally, the yield stress is at least about 20 Pa, 30 Pa, 40 Pa to about 2000 Pa. Likewise, the time to recover the yield stress after being sheared to flow at high shear or the shear experienced upon dispensing is as short as possible. For example, it is desirable that at least about 50% of the yield stress is recovered after being shear in fractions of second or at most about 1, 5 or even 10 seconds.

In an embodiment, the polymerizable composition may also be comprised of a multifunctional 1,1-dicarbonyl-1-alkene described above or a monomeric 1,1-dicarbonyl alkene described below. In another embodiment, the polymerizable composition may be comprised of an addition polymerizable compound such as those known in the art. Generally, it is desirable for the other addition polymerizable compound to undergo addition polymerization under similar conditions as the prepolymer. The addition polymerizable compound, for example, desirably has an electron deficient alkene. Examples of addition polymerizable compounds may include styrenic compounds, vinylidene aromatics, norbornene esters, allyl ethers, malemides, maleates, vinyl ethers, vinyl esters, triallyl isycyanurate and alkynes.

The monofunctional 1,1-dicarbonyl alkene may have a structure as shown below in Formula I, where X¹ and X² are an oxygen atom or a direct bond, and where R¹ and R² are each hydrocarbyl groups that may be the same or different. Both X¹ and X² may be oxygen atoms, such as illustrated in Formula IIA, one of X¹ and X² may be an oxygen atom and the other may be a direct bond, such as shown in Formula IIB, or both X¹ and X² are direct bonds, such as illustrated in Formula IIC. The 1,1-dicarbonyl alkene compounds used herein may have all ester groups (such as illustrated in Formula IIA), all keto groups (such as illustrated in Formula IIC) or a mixture thereof (such as illustrated in Formula IIB). Compounds with all ester groups may be preferred in some applications due to the flexibility of synthesizing a variety of such compounds.

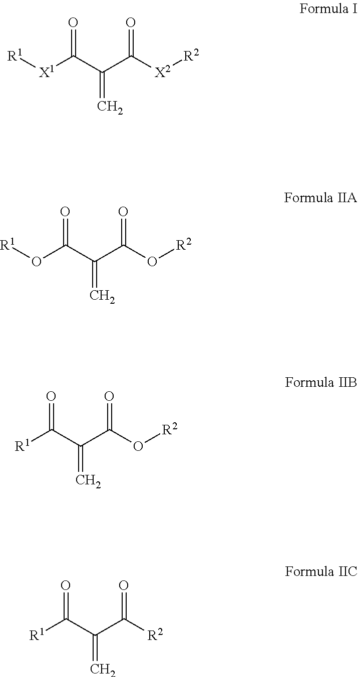

Heteroatom means nitrogen, oxygen, sulfur and phosphorus, more preferred heteroatoms include nitrogen and oxygen. Hydrocarbyl as used herein refers to a group containing one or more carbon atom backbones and hydrogen atoms, which may optionally contain one or more heteroatoms. Where the hydrocarbyl group contains heteroatoms, the heteroatoms may form one or more functional groups well known to one skilled in the art. Hydrocarbyl groups may contain cycloaliphatic, aliphatic, aromatic or any combination of such segments. The aliphatic segments can be straight or branched. The aliphatic and cycloaliphatic segments may include one or more double and/or triple bonds. Included in hydrocarbyl groups are alkyl, alkenyl, alkynyl, aryl, cycloalkyl, cycloalkenyl, alkaryl and aralkyl groups. Cycloaliphatic groups may contain both cyclic portions and noncyclic portions. Hydrocarbylene means a hydrocarbyl group or any of the described subsets having more than one valence, such as alkylene, alkenylene, alkynylene, arylene, cycloalkylene, cycloalkenylene, alkarylene and aralkylene. One or both hydrocarbyl groups may consist of one or more carbon atoms and one or more hydrogen atoms. As used herein percent by weight or parts by weight refer to, or are based on, the weight of the solution composition unless otherwise specified.

A preferred class of monofunctional 1,1-dicarbonyl alkene compounds is methylene malonates, the core structural unit/formula for which is shown below:

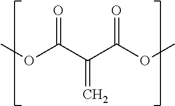

The term “monofunctional” refers to 1,1-dicarbonyl alkene compounds or a methylene malonate having only one core unit or one carbon carbon double bond. The term “difunctional” refers to 1,1-dicarbonyl alkene compounds or a methylene malonate having two core formulas bound through a hydrocarbyl linkage between one oxygen atom on each of two core formulas. The term “multifunctional” refers to 1,1-dicarbonyl alkene compounds or methylene malonates having more than one core formula which forms a chain through a hydrocarbyl linkage between one oxygen atom on each of two adjacent core formulas.

The monofunctional 1,1-dicarbonyl alkene may be a 1,1-diester-1-alkene. As used herein, diester refers to any compound having two ester groups. A 1,1-diester-1-alkene is a compound that contains two ester groups and a double bond bonded to a single carbon atom referred to as the one carbon atom. Dihydrocarbyl dicarboxylates are diesters having a hydrocarbylene group between the ester groups wherein a double bond is not bonded to a carbon atom which is bonded to two carbonyl groups of the diester.

The hydrocarbyl groups (e.g., R¹ and R²), each may be as described above.

The monofunctional 1,1-dicarbonyl alkene may be comprised of one or more of methyl propyl methylene malonate, dihexyl methylene malonate, di-isopropyl methylene malonate, butyl methyl methylene malonate, ethoxyethyl ethyl methylene malonate, methoxyethyl methyl methylene malonate, hexyl methyl methylene malonate, dipentyl methylene malonate, ethyl pentyl methylene malonate, methyl pentyl methylene malonate, ethyl ethylmethoxy methylene malonate, ethoxyethyl methyl methylene malonate, butyl ethyl methylene malonate, dibutyl methylene malonate, diethyl methylene malonate (DEMM), diethoxy ethyl methylene malonate, dimethyl methylene malonate, di-N-propyl methylene malonate, ethyl hexyl methylene malonate, methyl fenchyl methylene malonate, ethyl fenchyl methylene malonate, 2 phenylpropyl ethyl methylene malonate, 3 phenylpropyl ethyl methylene malonate, ethyl cyclohexyl methylene malonate, and dimethoxy ethyl methylene malonate.

The amount of 1,1-dicarbonyl alkene present in the polymerizable composition may be any amount to realize a desired property of the resultant polymer. For example, the amount of the 1,1-dicarbonyl alkene may be present in an amount of 0.1%, 1% to about 10%, 15% or 20% by weight of the prepolymer and the 1,1-dicarbonyl alkene or the total weight of the polymerizable composition. Similarly, the amount of another addition polymerizable compound (e.g., styrene) may also be present in such amounts in the polymerizable composition.

The 1,1-dicarbonyl alkene may be produced and purified by the methods described in U.S. Pat. Nos. 8,609,8985; 8,884,051; 9,108,914 and 9,518,001 and Int. Pub. WO 2017/197212. Examples of such 1,1-dicarbonyl alkenes are available under the tradenames CHEMILIAN and FORZA and include, for example, methylene malonate, dihexyl methylene malonate, dicyclohexyl methylene malonate and multifunctional polyester methylene malonates available from Sirrus, Inc., Loveland, Ohio (Nippon Shokubai, Japan).

In some embodiments, filler may be added to form the polymerizable composition. The filler may also act as a rheological modifier. The filler may be a carbon black have an oil absorption (OAN) of about 80 to 200 ccs per 100 grams. Preferably, the oil absorption of the carbon is at least about 90, more preferably at least about 100, and most preferably at least about 110 to preferably at most about 180, more preferably at most about 165 and most preferably at most about 150 ccs/100 grams.

In an embodiment, the prepolymer may be further mixed with another multifunctional Michael addition donor (MAD) compound that is not a thiol and that may react at conditions above ambient conditions or in the presence of an added catalyst. Examples of such groups include amines, carboxylic acids and hydroxyls, phosphonic or phosphoric acid, sulfonic or sulfuric acid moieties. These multifunctional MAD compounds typically will have a higher Mw such as 1000, or 2000 to 30,000 g/moles.

The polymer formed by polymerizing the prepolymer, functionalized prepolymer or the polymerizable composition made therefrom may be comprised of essentially addition polymerized alkene bonds and the reaction linkage of the thiol of the polythiol and alkene of the compounds. Or as just described, other linkages may arise in the polymerizable composition polymerization such as Michael addition linkages between the other Michael addition donor compounds such as a hydroxyl compound (e.g., polyols).

In another embodiment, the polymer may be formed by polymerizing the prepolymer by further addition of a separately added polyfunctional thiol as a curing additive or compound containing a thiol and another Michael addition functional group as described above (e.g., hydroxyl). In this embodiment, the curing may be performed at ambient temperature (˜20 to 25° C.) or elevated temperatures as well as in the presence of an anionic initiator described herein.

The aforementioned prepolymers or polymerizable compositions made therefrom may be polymerized by heating to a polymerization temperature wherein at least a portion of the alkenes remaining in the prepolymer undergo addition polymerization. The polymerization temperature maybe any useful to initiate the desired polymerization. Generally, the temperature is above ambient to about the decomposition temperature of the underlying prepolymer or other addition polymerizable compound. Typically, the temperature is from about 40° C., 60° C., 80° C. or 100° C. to about 250° C., 200° C., 180° C. or 150° C.

To facilitate anionic addition, an anionic initiator may be added to prior to or during the polymerization. A latent catalyst may be added if a one component polymerizable composition is desired. The anionic initiator may be any suitable anionic initiator that initiates anionic polymerization upon contact with the prepolymer.

A wide variety of anionic initiators may be used including most nucleophilic initiators capable of initiating anionic polymerization. Exemplary initiators include alkali metal salts, alkaline earth metal salts, ammonium salts, amines, halides (halogen containing salts), metal oxides, and mixtures containing such salts or oxides. Exemplary anions for such salts include anions based on halogens, acetates, benzoates, sulfur, carbonates, silicates and the like. Examples of anionic initiators may include glass beads having a basic constituent such as soda-lime silica glass, ceramic beads (comprised of various metals, nonmetals and metalloid materials), clay minerals (including hectorite clay and bentonite clay), and ionic compounds such as sodium silicate, sodium benzoate, and calcium carbonate. Additional suitable anionic initiators are also disclosed in U.S. Pat. No. 9,181,365 (col. 9, lines 45-57) and U.S. Pat. No. 9,334,430 incorporated herein by reference.

The amine anionic initiator may be any primary, secondary or tertiary amine and may be an alkyl or substituted alkyl amine. The alkyl or substituted alky may be any hydrocarbyl group typically having from 1 to 30 carbons and 1 to 6 heteroatoms such as sulfur and oxygen. In an embodiment the amines include, for example, substituted (e.g., 1 or 2 heteroatoms) or unsubstituted C₁-C₅ mono- and diamines, aromatic amines, and mixtures thereof. The amine compounds may have molecular weights from about 50 to about 10,000. In general, the lower molecular weight amines may be desired, for example, to enhance ease of mixing. Lower molecular weight amines generally have a molecular weight of less than about 1500 g/moles or less than about 1000 g/mol. Examples of amines include ethylamine, diethylamine, triethylamine, ethanolamine, diethanolamine, triethanolamine, butylamine, dibutylamine, tributylamine, butanolamine, dibutanolamine, tributanolamine, propanolamine, dipropanolamine, tripropanolamine, propylamine, dipropylamine, tripropylamine, ethylenediamine, triethylenediamine, N,N-dimethylbenzylamine, isophoronediamine, ethyl 1-methyl-3-pi peridi neca rboxylate, ethyl-1-methyl-4-piperidinecarboxylate, bis(2,2-morpholylethyl)ether (DMDEE), and mixtures thereof.

The anionic initiator may be employed in any amount sufficient to facilitate the anionic polymerization to the extent desired. The anionic initiator typically is used in an amount of about 0.01 to 20%, or about 0.1 or 0.25 to 8%, of about 0.5 to 5% or about 0.75 to 2% by weight of the prepolymer or the prepolymer and any other component mixed with the 1,1-dicarbonyl substituted alkene (i.e., weight of the polymerizable composition).

In some embodiments the anionic initiator may be latent to assist, wherein each of the components to be mixed with the prepolymer is contained in one package together that is activated, for example, upon discharge from the package and application of some force. For example, the composition may be discharge and mixed by a static or dynamic mixer activating the latent initiator. In other embodiments the anionic initiator may be activated by irradiating, heating or exposing to a solvent, for example that dissolves a coating or encapsulant enveloping the initiator.

The prepolymer or functionalized prepolymer may be contained separately and brought together and mixed with other ingredients (e.g., anionic initiator, multifunctional MAD compound, fillers dyes and the like) to polymerize the prepolymer to form an article. Suitable separate packaging or delivery systems may include those known in the art. Illustrative examples include those involving separate rigid tubes in which each material is dispensed by a separate plunger and mixed upon exiting using a static or dynamic mixing nozzles such as described by Craig Blum, Two Component Adhesive Cartridge Systems, FAST, July 2008. In another illustration, two or more compartmented sausage containers may be used such as described in U.S. Pat. Nos. 4,009,778; 4,126,005; 4,227,612; 6,129,244; 8,313,006 and 9,821,512.

The latent anionic initiator may be any employing an encapsulant. An illustrative example those described in U.S. Pat. No. 9,334,430 incorporated herein by reference in its entirety for all purposes.

In another embodiment, the anionic initiator may be a latent base such as those that absorb radiation such as radiation in the UV or visible region and forms a base or nucleophilic anionic initiator

Examples of photolatent bases include photocleavable carbamates (e.g., 9-xanthenylmethyl, fluorenylmethyl, 4-methoxyphenacyl, 2,5-dimethylphenacyl, benzyl, and others), which have been shown to generate primary or secondary amines after photochemical cleavage. Other photolatent bases which generate primary or secondary amines include certain O-acyloximes, sulfonamides, and formamides. Acetophenones, benzophenones, and acetonaphthones bearing quaternary ammonium undergo photocleavage to generate tertiary amines in the presence of a variety of counter cations (borates, dithiocarbamates, and thiocyanates). Examples of these photolatent ammonium salts are N-(benzophenonemethyl)tri-N-alkyl ammonium tetraarylborates or alkyltriarylborates or dialkyldiarylborates. Sterically hindered α-aminoketones generate tertiary amines. Exemplary photolatent bases useful for practicing the present disclosure include 5-benzyl-1,5-diazabicyclo[4.3.0]nonane, 5-(anthracen-9-yl-methyl)-1,5-diaza[4.3.0]nonane, 5-(2′-nitrobenzyl)-1,5-diazabicyclo[4.3.0]nonane, 5-(4′-cyanobenzyl)-1,5-diazabicyclo[4.3.0]nonane, 5-(3′-cyanobenzyl)-1,5-diazabicyclo[4.3.0]nonane, 5-(anthraquinon-2-yl-methyl)-1,5-diaza[4.3.0]nonane, 5-(2′-chlorobenzyl)-1,5-diazabicyclo[4.3.0]nonane, 5-(4′-methylbenzyl)-1,5-diazabicyclo[4.3.0]nonane, 5-(2′,4′,6′-trimethylbenzyl)-1,5-diazabicyclo[4.3.0]nonane, 5-(4′-ethenylbenzyl)-1,5-diazabicyclo[4.3.0]nonane, 5-(3′-trimethylbenzyl)-1,5-diazabicyclo[4.3.0]nonane, 5-(2′,3′-dichlorobenzyl)-1,5-diazabicyclo[4.3.0]nonane, 5-(naphth-2-yl-methyl-1,5-diazabicyclo[4.3.0]nonane, 1,4-bis(1,5-diazabicyclo[4.3.0]nonanylmethyl)benzene, 8-benzyl-1,8-diazabicyclo[5.4.0]undecane, 8-benzyl-6-methyl-1,8-diazabicyclo[5.4.0]undecane, 9-benzyl-1,9-diazabicyclo[6.4.0]dodecane, 10-benzyl-8-methyl-1,10-diazabicyclo[7.4.0]tridecane, 11-benzyl-1,11-diazabicyclo[8.4.0]tetradecane, 8-(2′-chlorobenzyl)-1,8-diazabicyclo[5.4.0]undecane, 8-(2′,6′-dichlorobenzyl)-1,8-diazabicyclo[5.4.0]undecane, 4-(diazabicyclo[4.3.0]nonanylmethyl)-1,1′-biphenyl, 4,4′-bis(diazabicyclo[4.3.0]nonanylmethyl)-11′-biphenyl, 5-benzyl-2-methyl-1,5-diazabicyclo[4.3.0]nonane, 5-benzyl-7-methyl-1,5,7-triazabicyclo[4.4.0]decane, and combinations thereof.

An example of a photolatent base is available from BASF under the trade designation “CGI-90”, which is reported to be 5-benzyl-1,5-diazabicyclo[4.3.0]nonane (see, e.g., WO 2014/176490 (Knapp et al.)), which generates 1,5-diazabicyclo[4.3.0]non-5-ene (DBN) upon exposure to radiation (see, e.g., US2013/0345389 (Cai et al), 2-benzyl-1-(3,5-dimethoxyphenyl)-2-(dimethylamino)butan-1-one available from BASF under the trade designation CGI 277, p-(Ethylthio)phenyl methylcarbamate and 6-Nitroveratryl chloroformate diethyl amine.

When using a photolatent base, the polymerizable composition may also include a photosensitizer. Photosensitizers are compounds when used in conjunction with a photolatent base improve or allow the accelerates the activation of the latent anionic initiator or allows for the activation at longer wavelengths than the absorbance of the photolatent base. A photosensitizer may be a compound having an absorption spectrum that overlaps or closely matches the emission spectrum of the radiation source to be used and that can, for example, improve the energy transfer to the photolatent base. Exemplary classes of photosensitizers include aromatic carbonyl compounds, for example benzophenone, thioxanthone, anthraquinone and 3-acylcoumarin derivatives or dyes such as eosine, rhodamine and erythrosine dyes. Additional exemplary photoinitiators include: thioxanthones, such as thioxanthone, 2-isopropylthioxanthone, 2-chlorothioxanthone, 1-chloro-4-propoxythioxanthone, 2-dodecylthioxanthone, 2,4-diethylthioxanthone, 2,4-dimethylthioxanthone, 1-methoxycarbonylthioxanthone, 2-ethoxycarbonylthioxanthone, 3-(2-methoxyethoxycarbonyl)-thioxanthone, 4-butoxycarbonylthioxanthone, 3-butoxycarbonyl-7-methylthioxanthone, 1-cyano-3-chlorothioxanthone, 1-ethoxycarbonyl-3-chlorothioxanthone, 1-ethoxycarbonyl-3-ethoxythioxanthone, 1-ethoxycarbonyl-3-aminothioxanthone, 1-ethoxycarbonyl-3-phenylsulfurylthioxanthone, 3,4-di-[2-(2-methoxyethoxy)ethoxycarbonyl]-thioxanthone, 1,3-dimethyl-2-hydroxy-9H-thioxanthen-9-one 2-ethylhexylether, 1-ethoxycarbonyl-3-(1-methyl-1-morpholinoethyl)-thioxanthone, 2-methyl-6-dimethoxymethyl-thioxanthone, 2-methyl-6-(1,1-dimethoxybenzyl)-thioxanthone, 2-morpholinomethylthioxanthone, 2-methyl-6-morpholinomethylthioxanthone, N-allylthioxanthone-3,4-dicarboximide, N-octylthioxanthone-3,4-dicarboximide, N-(1,1,3,3-tetramethylbutyl)-thioxanthone-3,4-dicarboximide, 1-phenoxythioxanthone, 6-ethoxycarbonyl-2-methoxythioxanthone, 6-ethoxycarbonyl-2-methylthioxanthone, thioxanthone-2-carboxylic acid polyethyleneglycol ester, 2-hydroxy-3-(3,4-dimethyl-9-oxo-9H-thioxanthon-2-yloxy)-N,N,N-trimethyl-1-propanaminium chloride; 2. Benzophenones, such as benzophenone, 4-phenyl benzophenone, 4-methoxy benzophenone, 4,4′-dimethoxy benzophenone, 4,4′-dimethyl benzophenone, 4,4′-dichlorobenzophenone 4,4′-bis(dimethylamino)-benzophenone, 4,4′-bis(diethylamino)benzophenone, 4,4′-bis(methylethylamino)benzophen-one, 4,4′-bis(p-isopropylphenoxy)benzophenone, 4-methyl benzophenone, 2,4,6-trimethyl-benzophenone, 4-(4-methylthiophenyl)-benzophenone, 3,3′-dimethyl-4-methoxy benzophenone, methyl-2-benzoylbenzoate, 4-(2-hydroxyethylthio)-benzophenone, 4-(4-tolylthio)-benzophenone, 1-[4-(4-benzoyl-phenylsulfanyl)-phenyl]-2-methyl-2-(toluene-4-sulfonyl)-propan-1-one, 4-benzoyl-N,N,N-trimethylbenzenemethanaminium chloride, 2-hydroxy-3-(4-benzoylphenoxy)-N,N,N-trimethyl-1-propanaminium chloride monohydrate, 4-(13-acryloyl-1,4,7,10,13-pentaoxamidecyl)-benzophenone, 4-benzoyl-N, N-dimethyl-N-[2-(1-oxo-2-propenyl)oxy]ethyl-benzenemethanaminium chloride; Coumarins, such as Coumarin 1, Coumarin 2, Coumarin 6, Coumarin 7, Coumarin 30, Coumarin 102, Coumarin 106, Coumarin 138, Coumarin 152, Coumarin 153, Coumarin 307, Coumarin 314, Coumarin 314T, Coumarin 334, Coumarin 337, Coumarin 500, 3-benzoyl coumarin, 3-benzoyl-7-methoxycoumarin, 3-benzoyl-5,7-dimethoxycoumarin, 3-benzoyl-5,7-dipropoxycoumarin, 3-benzoyl-6,8-dichlorocoumarin, 3-benzoyl-6-chloro-coumarin, 3,3′-carbonyl-bis[5,7-di(propoxy)coumarin], 3,3′-carbonyl-bis(7-methoxycoumarin), 3,3′-carbonyl-bis(7-diethylamino-coumarin), 3-isobutyroylcoumarin, 3-benzoyl-5,7-dimethoxy-coumarin, 3-benzoyl-5,7-diethoxy-coumarin, 3-benzoyl-5,7-dibutoxycoumarin, 3-benzoyl-5,7-di(methoxyethoxy)-coumarin, 3-benzoyl-5,7-di(allyloxy)coumarin, 3-benzoyl-7-dimethylaminocoumarin, 3-benzoyl-7-diethylaminocoumarin, 3-isobutyroyl-7-dimethylaminocoumarin, 5,7-dimethoxy-3-(1-naphthoyl)-coumarin, 5,7-diethoxy-3-(1-naphthoyl)-coumarin, 3-benzoylbenzo[f]coumarin, 7-diethylamino-3-thienoylcoumarin, 3-(4-cyanobenzoyl)-5,7-dimethoxycoumarin, 3-(4-cyanobenzoyl)-5,7-dipropoxycoumarin, 7-dimethylamino-3-phenylcoumarin, 7-diethylamino-3-phenylcoumarin, the coumarin derivatives disclosed in JP 09-179299-A and JP 09-325209-A, for example 7-[{4-chloro-6-(diethylamino)-S-triazine-2-yl}amino]-3-phenylcoumarin; 4. 3-(aroylmethylene)-thiazolines, such as 3-methyl-2-benzoylmethylene-β-naphthothiazoline, 3-methyl-2-benzoylmethylene-benzothiazoline, 3-ethyl-2-propionylmethylene-β-naphthothiazoline; Rhodanines, such as 4-dimethylaminobenzalrhodanine, 4-diethylaminobenzalrhodanine, 3-ethyl-5-(3-octyl-2-benzothiazolinylidene)-rhodanine; other Compounds, such as acetophenone, 3-methoxyacetophenone, 4-phenylacetophenone, benzil, 4,4′-bis(dimethylamino)benzil, 2-acetylnaphthalene, 2-naphthaldehyde, dansyl acid derivatives, 9,10-anthraquinone, anthracene, pyrene, aminopyrene, perylene, phenanthrene, phenanthrenequinone, 9-fluorenone, dibenzosuberone, curcumin, xanthone, thiomichler's ketone, α-(4-dimethylaminobenzylidene) ketones, e.g. 2,5-bis(4-diethylaminobenzylidene)cyclopentanone, 2-(4-dimethylamino-benzylidene)-indan-1-one, 3-(4-dimethylamino-phenyl)-1-indan-5-yl-propenone, 3-phenylthiophthalimide, N-methyl-3,5-di(ethylthio)-phthalimide, N-methyl-3,5-di(ethylthio)phthalimide, phenothiazine, methylphenothiazine, amines, e.g. N-phenylglycine, ethyl 4-dimethylaminobenzoate, butoxyethyl 4-dimethylaminobenzoate, 4-dimethylaminoacetophenone, triethanolamine, methyldiethanolamine, dimethylaminoethanol, 2-(dimethylamino)ethyl benzoate, poly(propylenegylcol)-4-(dimethylamino)benzoate. The weight ratio of photolatent anioinic initiators (e.g., base) to the weight of photosensitizers may be range from 0.5/1 to 10/1 or 1/1 to 5/1.

The polymerizable compositions may comprise one or more free radical stabilizers. The one or more free radical stabilizers may be present in sufficient amount to prevent undesired addition polymerization. The free radical polymerization stabilizers may be present in an amount of about 10 ppm or less based on the weight of the total composition (the 1,1-dicarbonyl substituted alkene mixed with other ingredients) or the 1,1-dicarbonyl substituted alkene itself whether mixed with other ingredients or not, about 100 ppm by weight or greater, or about 1000 ppm by weight or greater. The free radical polymerization stabilizers may be present in an amount of about 10,000 ppm by weight or less based on the weight of the total composition, about 8000 ppm by weight or less, or about 5000 ppm by weight or less. The free radical inhibitors that may be used include: tocopherol (e.g., including vitamin E), 4-tert-Butylpyrocatechol; tert-Butylhydroquinone; 1,4-Benzoquinone; 6-tert-Butyl-2,4-xylenol; 2-tert-Butyl-1,4-benzoquinone; 2,6-Di-tert-butyl-p-cresol; 2,6-Di-tert-butylphenol; Hydroquinone; 4-Methoxyphenol; Phenothiazine; 2,2′-methylenebis(6-tert-butyl-4-methylphenol) or a combination thereof. Free radical stabilizers preferably include phenolic compounds (e.g., 4-methoxyphenol, mono methyl ether of hydroquinone (“MeHQ”) butylated hydroxytoluene (“BHT”)). Stabilizer packages for 1,1-disubstituted alkenes are disclosed in U.S. Pat. Nos. 8,609,885 and 8,884,051, each incorporated by reference. Additional free radical polymerization inhibitors are disclosed in U.S. Pat. No. 6,458,956, incorporated by reference.

The polymerizable compositions may contain a filler in certain embodiments such as printing dyes or additive manufacturing techniques such as polyjetting and inkjetting, which are described below. Examples of filler include talc, wollastonite, mica, clay, montmorillonite, smectite, kaolin, calcium carbonate, glass fibers, glass beads, glass balloons, glass milled fibers, glass flakes, carbon fibers, carbon flakes, carbon beads, carbon milled fibers, metal flakes, metal fibers, metal coated glass fibers, metal coated carbon fibers, metal coated glass flakes, silica, other ceramic particles, ceramic fibers, ceramic balloons, aramid particles, aramid fibers, polyacrylate fibers, graphite, and various whiskers such as potassium titanate whiskers, aluminum borate whiskers and basic magnesium sulfate whiskers. The fillers may be incorporated alone or in combination. The filler may be present in the polymerizable composition in any useful amount such as from about 1% to 90% depending on the particular application.

The prepolymer, functionalized prepolymer or polymerizable compositions made therefrom may be used to make polymers and articles that may be used in any number of applications. Exemplary applications include adhesives, sealants, films, coatings, components for optical fibers, potting and encapsulating materials for electronics, resins, molded articles, and the like.

To form the polymer or article from the prepolymer or functionalized prepolymer and each ingredient desired is mixed with the prepolymer or functionalized prepolymer to form a polymerizable composition. In an embodiment, the polymerizable composition is provided in at least two separate components prior to mixing as previously described. Desirably, the prepolymer or functionalized prepolymer is provided in a first component and if desired an anionic initiator is provided in another component. The composition may be provided in a singular container having a plurality of chambers that separates each of the components from reacting prior to mixing of the component. The components may be dispensed through a common orifice causing each of the ingredients of the composition to mix. To aid the mixing through the orifice, a static or dynamic mixer may be employed.

In another embodiment, the prepolymer or functionalized prepolymer is provided in one component or container together, wherein the anionic initiator is latent as described previously. The ingredients in the composition may be dispensed under sufficient mixing (mechanical force) such that the latent anionic initiator is activated and the prepolymer polymerizes forming the desired polymer or article. Alternatively, the prepolymer may be mixed with other ingredients as desired (form polymerizable composition), dispensed and then subjected to heating or irradiating to initiate polymerization.

When a photolatent anionic initiator (e.g., photolatent base) is used, the radiation source may be any suitable one such as those known in the art. Illustratively, the radiation is UV and the UV sources may be any suitable device such as those known in the art and include, for example, commercially available UV light emitting diodes (LEDs) and mercury lamps with or without filters.

In an embodiment a substrate is coated by the prepolymer or functionalized prepolymer or polymerizable composition made therefrom forming an article with polymer formed from the prepolymer or functionalized prepolymer adhered to the substrate. In another embodiment, the prepolymer or functionalized prepolymer or polymerizable composition made therefrom is interposed on two or more substrates and polymerized to adhere the two or more substrates together. In another embodiment, the prepolymer or functionalized prepolymer and any other ingredients may be dispensed, cast or injected into a mold and allowed to cure or react to form a shaped article. The dispensing to form a coated article or article comprised of two substrates adhered together by the composition's polymer reaction product may be any suitable dispensing method such as those known in the art (e.g., spraying, painting, caulk gunning, extruding and the like). The substrates may be any suitable substrate such as a ceramic, metal, metalloid, glass, plastic, wood, a composite of any of the aforementioned, or combination thereof.

In an embodiment, the prepolymer, functionalized prepolymer or polymerizable composition made therefrom may be used to form an article employing known forming techniques such as additive manufacturing techniques. Illustratively, the polymerizable composition may be comprised of a latent anionic initiator which are formed into an article by additive manufacturing methods such as photopolymerization 3D printing techniques. Stereolithography (SLA), where a UV laser beam is rastered upon a vat of the curable composition initiating polymerization and is built up layer by layer, which is illustrated by U.S. Pat. Nos. 4,575,330 and 5,256,340 may be used. Digital light processing (DLP) where an image is flashed at once upon a vat using projections of graphics using conventional light sources employing mirrors typically, with subsequent layers being built up by changing the image layer by layer (see, for example, U.S. Pat. Nos. 5,236,637 and 10,001,641 may be used. Continuous liquid interface production (CLIP) as described in U.S. Pat. No. 9,211,678 and Daylight polymer printing (DPP) as described in U.S. Pat. Appl. 2018/0141268 may also be used. Other exemplary 3D printing methods that may be used include those employing the polymerizable composition as a binder that is sprayed upon a powder bed that is then irradiated and layer built up to form the article (Binder Jetting “BJ” see for example U.S. Pat. No. 5,340,656) or within a printing ink that is irradiated for example layer by layer by using (polyjet or inkjet, see for example, U.S. Pat. Pub. 2018/0029291), or a thin sheet is irradiated with a subsequent sheet layer to the previous sheet and it being irradiated to laminate it to its previous sheet (Sheet lamination, “SL” see for example U.S. Pat. No. 5,876,550).

### Illustrative Embodiments

Embodiment 1. A method of forming a 1,1-dicarbonyl substituted-1-alkene prepolymer comprising:

a. mixing a multifunctional 1,1-dicarbonyl substituted-1-alkene having an average functionality of greater than 1 to 10 and a polythiol having an average functionality greater than 1 to about 6 at a ratio of thiols/carbon-carbon double bonds of 0.001 to 0.5 to form a mixture,

b. allowing the multifunctional 1,1-dicarbonyl substituted-1-alkene and polythiol to react in the presence of a strong acid at a concentration of 0.1 parts per million to 100 parts per million by weight of the mixture to form the 1,1-dicarbonyl substituted-1-alkene prepolymer.

Embodiment 2. The method of Embodiment 1, wherein the ratio of thiols/carbon-carbon double bonds is 0.01 to 0.4.

Embodiment 3. The method of Embodiment 2, wherein the ratio of thiols/alkenes is 0.1 to 0.3.

Embodiment 4. The method of any one of the preceding Embodiments, wherein the reaction is performed at ambient conditions and in the absence of any catalyst other than the strong acid.

Embodiment 5. The method any one of the preceding Embodiments, wherein the polythiol has a weight average molecular weight (Mw) of 200 to 30,000 g/mole.

Embodiment 6. The method of Embodiment 5, wherein the polythiol (Mw) is 2000 to 20,000 g/mole.

Embodiment 7. The method of any one of the preceding Embodiments, wherein the polythiol has an average functionality of about 1.5 to about 2.5.

Embodiment 8. The method of any one of the preceding Embodiments, wherein the prepolymer has a viscosity of 100 to 50,000 centipoise.

Embodiment 9. The method of any one of the preceding Embodiments, the prepolymer has a shelf life of at least about 4 months.

Embodiment 10. The method of any one of the preceding Embodiments wherein the prepolymer has an alkene number of 50 to 75.

Embodiment 11. The method of any one of the preceding Embodiments, wherein the prepolymer has an Mw of 1000 to 100,000 g/moles.

Embodiment 12. The method of any one of the preceding Embodiments, wherein the multifunctional 1,1-dicarbonyl substituted alkene is represented by:

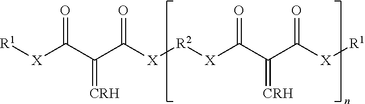

wherein X, X¹ and X² are an oxygen atom or a direct bond, and where R¹ and R² are each hydrocarbyl groups having from 1 to 30 carbons and R is hydrogen or a hydrocarbyl group having from 1 to 30 carbons, so long as at least one R is hydrogen.

Embodiment 13. The method of Embodiment 12, wherein X is an oxygen atom and R2 is the residue of a polyol.

Embodiment 14. The method of any one of Embodiments 1 to 13, wherein the 1,1-dicarbonyl substituted alkene is the reaction product of the transesterification of a polyol and methyl propyl methylene malonate, dihexyl methylene malonate, di-isopropyl methylene malonate, butyl methyl methylene malonate, ethoxyethyl ethyl methylene malonate, methoxyethyl methyl methylene malonate, hexyl methyl methylene malonate, dipentyl methylene malonate, ethyl pentyl methylene malonate, methyl pentyl methylene malonate, ethyl ethylmethoxy methylene malonate, ethoxyethyl methyl methylene malonate, butyl ethyl methylene malonate, dibutyl methylene malonate, diethyl methylene malonate (DEMM), diethoxy ethyl methylene malonate, dimethyl methylene malonate, di-N-propyl methylene malonate, ethyl hexyl methylene malonate, methyl fenchyl methylene malonate, ethyl fenchyl methylene malonate, 2 phenylpropyl ethyl methylene malonate, 3 phenylpropyl ethyl methylene malonate, ethyl cyclohexyl methylene malonate, and dimethoxy ethyl methylene malonate.

Embodiment 15. The method of any one of the preceding Embodiments, wherein the polythiol has a backbone that is comprised of an aromatic, aliphatic, combination of aliphatic and aromatic, polyether, polyester, polythioether, polysulfide or combination thereof.

Embodiment 16. A 1,1-dicarbonyl substituted-1-alkene prepolymer comprised of the reaction product of a multifunctional 1,1-dicarbonyl substituted-1-alkene and a polythiol, wherein the prepolymer is a liquid having an amount of a strong acid from 0.1 ppm to 100 ppm by weight of the prepolymer and strong acid.

Embodiment 17. The 1,1-dicarbonyl substituted-1-alkene prepolymer of Embodiment 16, wherein the prepolymer has a viscosity of 100 to 50,000 centipoise.

Embodiment 18. The prepolymer of either Embodiment 16 or 17, wherein the prepolymer has a shelf life of at least 3 months.

Embodiment 19. The prepolymer of Embodiment 18, wherein the shelf life is at least 6 months.

Embodiment 20. The prepolymer of any one of Embodiments 16 to 19, wherein the amount of strong acid is from 1 ppm to 50 ppm by weight of the prepolymer and strong acid.

Embodiment 21. The prepolymer of any one of Embodiments 16 to 20, wherein the strong acid has a pKa of less than 3.

Embodiment 22. The prepolymer of Embodiment 21, wherein the pKa is from 3 to −12.

Embodiment 23. The prepolymer of any one of Embodiments 16 to 22, wherein the strong acid is comprised of one or more of an inorganic acid, methane sulfonic acid, or para toluene sulfonic acid.

Embodiment 24. The prepolymer of Embodiment 23, wherein the strong acid is comprised of methane sulfonic acid.

Embodiment 25. A polymerizable composition comprised of the prepolymer of any one of Embodiments 16 to 24 and a further ingredient comprised of one or more of a filler, plasticizer, dye, an addition polymerizable monomer or oligomer that is different than the prepolymer, a polyfunctional Michael addition compound other than one containing a thiol, ultraviolet stabilizers, antioxidants, catalyst, or rheological modifiers.

Embodiment 26. The polymerizable composition of Embodiment 25, wherein the further ingredient is comprised of an addition polymerizable monomer or oligomer.

Embodiment 27. The polymerizable composition of Embodiment 26, wherein the addition polymerizable monomer or oligomer is comprised of a 1,1-dicarbonyl substituted-1-alkene.

Embodiment 28. The polymerizable composition of Embodiment 27, wherein the 1,1-dicarbonyl substituted alkene is represented by:

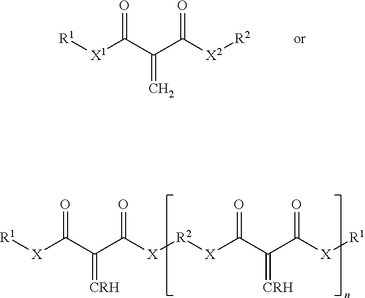

wherein X, X¹ and X² are an oxygen atom or a direct bond, and where R¹ and R² are each hydrocarbyl groups having from 1 to 30 carbons and R is hydrogen or a hydrocarbyl group having from 1 to 30 carbons, so long as at least one R is hydrogen.

Embodiment 28. The polymerizable composition of Embodiment 28, wherein the wherein the 1,1-dicarbonyl substituted alkene is represented by:

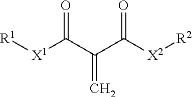

Embodiment 29. The polymerizable composition of any one of Embodiments 25 to 28, wherein the 1,1-dicarbonyl substituted alkene is comprised of one or more of methyl propyl methylene malonate, dihexyl methylene malonate, di-isopropyl methylene malonate, butyl methyl methylene malonate, ethoxyethyl ethyl methylene malonate, methoxyethyl methyl methylene malonate, hexyl methyl methylene malonate, dipentyl methylene malonate, ethyl pentyl methylene malonate, methyl pentyl methylene malonate, ethyl ethylmethoxy methylene malonate, ethoxyethyl methyl methylene malonate, butyl ethyl methylene malonate, dibutyl methylene malonate, diethyl methylene malonate (DEMM), diethoxy ethyl methylene malonate, dimethyl methylene malonate, di-N-propyl methylene malonate, ethyl hexyl methylene malonate, methyl fenchyl methylene malonate, ethyl fenchyl methylene malonate, 2 phenylpropyl ethyl methylene malonate, 3 phenylpropyl ethyl methylene malonate, ethyl cyclohexyl methylene malonate, and dimethoxy ethyl methylene malonate.

Embodiment 30. The polymerizable composition of any one of Embodiments 25 to 29, wherein the further addition polymerizable monomer is comprised of one or more of an acrylic, acrylate, methacrylate, vinyl ethers, vinyl aromatics, vinyl heteroaromatics, vinyl amides, vinyl lactones, vinyl lactams, vinyl carbonates, vinyl halides, vinyl amides, vinyl esters, olefins (e.g., alpha olefins), cycloalkenes, allyl group containing compounds, vinyl silanes, or vinyl sulfides.

Embodiment 31. An article comprised of a polymerized prepolymer of any one of Embodiments 16 to 24.

Embodiment 32. An article comprised of a polymerized polymerizable composition of any one of Embodiments 25 to 30.

Embodiment 33. The article of either Embodiment 31 or 32, wherein the article is a coating, adhesive, caulk, sealant, ink, molded article or additive manufactured article.

Embodiment 34. A method of forming a functionalized 1,1-dicarbonyl substituted-1-alkene prepolymer comprising:

a. mixing a multifunctional 1,1-dicarbonyl substituted-1-alkene having an average functionality of greater than 1 to 10 and a thiol having an additional functional group at a ratio of thiols/carbon-carbon double bonds of 0.001 to 0.5 to form a mixture,

b. allowing the multifunctional 1,1-dicarbonyl substituted-1-alkene and polythiol to react in the presence of a strong acid at a concentration of 0.1 parts per million to 100 parts per million by weight of the mixture to form the functionalized 1,1-dicarbonyl substituted-1-alkene prepolymer.

Embodiment 35. A functionalized 1,1-dicarbonyl substituted-1-alkene prepolymer comprised of the reaction product of a multifunctional 1,1-dicarbonyl substituted-1-alkene and a thiol having an additional functional group, wherein the prepolymer is a liquid having an amount of a strong acid from 0.1 ppm to 100 ppm (parts per million) by weight of the prepolymer and strong acid.

Embodiment 36. A polymerizable composition comprised of the functionalized 1,1-dicarbonyl substituted-1-alkene prepolymer of Embodiment 35 and a further ingredient comprised of one or more of a filler, plasticizer, dye, an addition polymerizable monomer or oligomer that is different than the prepolymer, a polyfunctional Michael addition compound other than one containing a thiol, ultraviolet stabilizers, antioxidants, catalyst, or rheological modifiers.

Embodiment 37. An article comprised of a polymerized functionalized 1,1-dicarbonyl substituted-1-alkene prepolymer of Embodiment 35.

Embodiment 38. An article comprised of a polymerized polymerizable composition of Embodiment 36.

### Examples

The following examples are provided to illustrate the curable compositions and the polymers formed from them, but are not intended to limit the scope thereof. All parts and percentages are by weight unless otherwise noted. Table 1 shows the ingredients used in the examples and comparative examples.

### Synthesis of Prepolymers

Prepolymers were synthesized by mixing the BDPES component and the thiol component in the absence of solvent in a FlackTek SpeedMixer at 1500 rpm for 5 minutes. The thiol addition reaction to the alkene proceeds at room temperature without additional catalyst, and the reaction was measured to be complete after 24 hours for all examples (as measured by FT-IR as the disappearance of the S-H stretching peak with an absorbance of 2600-2550 cm⁻¹).

Prepolymer Property Measurement

Viscosity was measured by a rheometer with 25 mm parallel plates. The rotation rate was set to 1.57 rad/s and the temperature was controlled at 25° C. Alkene number is the percentage of methylene malonate groups that remain unreacted. Note that the alkene number of pure BDPES used in these experiments is <100 because some of the methylene malonate groups have reacted during its synthesis. This quantity is measured by H-NMR using hexamethyldisiloxane as an internal standard. The dimer content is the molar content of BDPES with 2 repeat units. This quantity and the number average molecular weight (Mn) were measured by GPC. PMMA molecular weight standards were used for calibration of the GPC column.

Prepolymer Reactivity Measurement

Gel time tests were conducted to study the reactivity of the examples towards anionic polymerization. Gel time was determined as the elapsed time between mixing the prepolymer and the initiator and the point at which the mixture will no longer flow under its own weight when the cup is inverted. The prepolymers were mixed with the initiator solution in plastic cups by a FlackTek SpeedMixer at 1500 rpm for 1 minute. For each example, the initiator solution was a mixture determined by molar ratio of neodecanoic acid and N,N-dimethylbenzylamine.

Cured Polymer Property Measurement

The prepolymer examples were cured into polymer films. For each example, 5 wt % of a mixture of neodecanoic acid and N,N-dimethylbenzylamine (DMBA) was used as the initiator for anionic polymerization. After mixing the prepolymer examples with initiator, they were cast into a mold consisting of two glass panels separated by spacers with 0.1 mm thickness. The mixtures remained in the molds for 1 day, allowing them to cure into polymer films. After one day, the films were removed from the molds and cut into strips 19 mm wide. 1 week after preparing the films, they were tested for tensile properties and C═C double bond conversion. C═C double bond conversion was measured by FT-IR as the disappearance of the absorbance of 830-770 cm⁻¹. Tensile properties were measured on an Instron with a 1 kN load cell. The tensile tests consisted of mounting the pre-cut strips with 40 mm length between the clamps and applying a strain of 0.2 in/minute until the samples breaks. Each data point listed in Table 2 for tensile strength and elongation represents an average of 5 tensile tests.

TABLE 1

| | Acronyms used | | |
|---|---|---|---|
| Component | herein | Company | Description |
| methylene | BDPES | Sirrus | Monomer: obtained from |
| malonate | | | transesterification of |
| polyester | | | 1,4-butanediol and |
| | | | diethyl methylene |
| | | | malonate |
| Ethylene glycol | GDMA | Sigma | Polythiol: |
| dimercaptoacetate | | | functionality = 2 |
| 3-Mercapto- | MPAcid | Sigma | Monothiol: with a |
| propionic | | | carboxylic acid |
| acid | | | functionality |
| N,N-dimethyl- | DBMA | Sigma | Initiator for anionic |
| benzylamine | | | polymerization |
| Neodecanoic acid | Versatic | Hexion | Chain transfer |
| | acid 10 | | agent/inhibitor for |
| | | | anionic polymerization |

TABLE 2

| | Comp. Ex. 1 | Example 1 | Example 2 | Example 3 |
|---|---|---|---|---|
| Prepolymer Composition (wt %) | | | | |
| BDPES | 100 | 95 | 90 | 90 |
| GDMA | 0 | 5 | 10 | 0 |
| MPAcid | 0 | 0 | 0 | 10 |
| Prepolymer Properties | | | | |
| Viscosity at | 208 | 535 | 1629 | 988 |
| 25 C (cP) | | | | |
| Alkene # | 81.1 | 68.3 | 57.5 | 57.1 |
| Dimer Content | 24.2 | 19.2 | 14.0 | 14.1 |
| (mol %) | | | | |
| Mn (Da) | 692 | 835 | 1096 | 839 |
| Prepolymer Reactivity | | | | |
| Gel time for 5 | 19 | 50 | 82 | >1200 |
| wt % 75:1 by | | | | |
| mole neodecanoic | | | | |
| acid:DMBA | | | | |
| initiator (min) | | | | |
| Gel time for | <1 | <1 | <1 | 3.5 |
| 5 wt % 1:1 by | | | | |
| mole neodecanoic | | | | |
| acid:DMBA | | | | |
| initiator (min) | | | | |
| Cured Polymer Properties | | | | |
| Wt % Initiator | 5 | 5 | 5 | 5 |
| Double Bond | 64.0 | | 72.7 | 85.5 |
| Conversion (%) | | | | |
| Tensile | 2660 | | 3040 | Not |
| Strength (psi) | | | | Determined* |
| Tensile | 1.8 | | 16.1 | Not |
| Elongation (%) | | | | Determined* |

*Polymer was too soft to measure by the technique described herein.

1. A method of forming a 1,1-dicarbonyl substituted-1-alkene prepolymer comprising:
a. mixing a multifunctional 1,1-dicarbonyl substituted-1-alkene having an average functionality of greater than 1 to 10 and a polythiol having an average functionality greater than 1 to about 6 at a ratio of thiols/carbon-carbon double bonds of 0.001 to 0.5 to form a mixture,
b. allowing the multifunctional 1,1-dicarbonyl substituted-1-alkene and polythiol to react in the presence of a strong acid at a concentration of 0.1 parts per million to 100 parts per million by weight of the mixture to form the 1,1-dicarbonyl substituted-1-alkene prepolymer.

2. The method of claim 1, wherein the ratio of thiols/carbon-carbon double bonds is 0.01 to 0.4.

3. The method of claim 1, wherein the reaction is performed at ambient conditions and in the absence of any catalyst other than the strong acid.

4. The method of claim 1, wherein the polythiol has an average functionality of about 1.5 to about 2.5.

5. The method of claim 1, wherein the prepolymer has a viscosity of 100 to 50,000 centipoise.

6. The method of claim 1 wherein the prepolymer has an alkene number of 50 to 75.

7. The method of claim 1, wherein the prepolymer has an M_{w} of 1000 to 100,000 g/moles.

8. The method of claim 1, wherein the multifunctional 1,1-dicarbonyl substituted alkene is represented by: 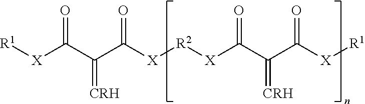
wherein X, X¹ and X² are an oxygen atom or a direct bond, and where R¹ and R² are each hydrocarbyl groups having from 1 to 30 carbons and R is hydrogen or a hydrocarbyl group having from 1 to 30 carbons, so long as at least one R is hydrogen.

9. A 1,1-dicarbonyl substituted-1-alkene prepolymer comprised of the reaction product of a multifunctional 1,1-dicarbonyl substituted-1-alkene and a polythiol, wherein the prepolymer is a liquid having an amount of a strong acid from 0.1 ppm to 100 ppm by weight of the prepolymer and strong acid.

10. The prepolymer of any one of claim 9, wherein the strong acid has a pKa of less than 3.

11. A polymerizable composition comprised of the prepolymer of claim 9 and a further ingredient comprised of one or more of a filler, plasticizer, dye, an addition polymerizable monomer or oligomer that is different than the prepolymer, a polyfunctional Michael addition compound other than one containing a thiol, ultraviolet stabilizers, antioxidants, catalyst, or rheological modifiers.

12. The polymerizable composition of claim 11, wherein the further ingredient is comprised of an addition polymerizable monomer or oligomer.

13. The polymerizable composition of claim 12, wherein the addition polymerizable monomer or oligomer is comprised of a 1,1-dicarbonyl substituted-1-alkene.

14. The polymerizable composition of claim 12, wherein the addition polymerizable monomer is comprised of one or more of an acrylic, acrylate, methacrylate, vinyl ethers, vinyl aromatics, vinyl heteroaromatics, vinyl amides, vinyl lactones, vinyl lactams, vinyl carbonates, vinyl halides, vinyl amides, vinyl esters, olefins, cycloalkenes, allyl group containing compounds, vinyl silanes, or vinyl sulfides

15. An article comprised of a polymerized prepolymer of claim 11.

16. (canceled)

17. A functionalized 1,1-dicarbonyl substituted-1-alkene prepolymer comprised of the reaction product of a multifunctional 1,1-dicarbonyl substituted-1-alkene and a thiol having an additional functional group, wherein the prepolymer is a liquid having an amount of a strong acid from 0.1 ppm to 100 ppm (parts per million) by weight of the prepolymer and strong acid.

18. A polymerizable composition comprised of the functionalized 1,1-dicarbonyl substituted-1-alkene prepolymer of claim 17 and a further ingredient comprised of one or more of a filler, plasticizer, dye, an addition polymerizable monomer or oligomer that is different than the prepolymer, a polyfunctional Michael addition compound other than one containing a thiol, ultraviolet stabilizers, antioxidants, catalyst, or rheological modifiers.

19. (canceled)

20. An article comprised of a polymerized polymerizable composition of claim 18.
